SEQUENCE LISTING (11 sequences)

SEQ ID NO: 1 - protein, 11 aa, Mycobacterium bovis
RTRVKVVKNKC

SEQ ID NO: 2 - protein, 12 aa, Mycobacterium bovis
HNCSPPFKQAEF

SEQ ID NO: 3 - protein, 442 aa, Mycobacterium bovis
KCLAEGTRIFDPVTGTTHRIEDVVDGRKPIHVVAAAKDGTLHARPVVSWFDQGTRDVIGLRIAGGAIVWATPDHKVLTEYGWRAAGELRKGDRVAQPRRFDGFGDSAPIPADHARLLGYLIGDGRDGWVGGKTPINFINVQRALIDDVTRIAATLGCAAHPQGRISLAIAHRPGERNGVADLCQQAGIYGKLAWEKTIPNWFFEPDIAADIVGNLLFGLFESDGWVSREQTGALRVGYTTTSEQLAHQIHWLLLRFGVGSTVRDYDPTQKRPSIVNGRRIQSKRQVFEVRISGMDNVTAFAESVPMWGPRGAALIQAIPEATQGRRRGSQATYLAAEMTDAVLNYLDERGVTAQEAAAMIGVASGDPRGGMKQVLGASRLRRDRVQALADALDDKFLHDMLAEELRYSVIREVLPTRRARTFDLEVEELHTLVAEGVVVHNC

SEQ ID NO: 4 - protein, 13 aa, Mycobacterium bovis
CLAEGTRIFDPVT

SEQ ID NO: 5 - protein, 14 aa, Mycobacterium bovis
GAIVWATPDHKVLT

SEQ ID NO: 6 - protein, 9 aa, Mycobacterium bovis
LLGYLIGDG

SEQ ID NO: 7 - protein, 8 aa, Mycobacterium bovis
EKTIPNWF

SEQ ID NO: 8 - protein, 10 aa, Mycobacterium bovis
LLFGLFESDG

SEQ ID NO: 9 - protein, 19 aa, Mycobacterium bovis
TTSEQLAHQIHWLLLRFGV

SEQ ID NO: 10 - protein, 14 aa, Mycobacterium bovis
RTFDLEVEELHTLV

SEQ ID NO: 11 - protein, 8 aa, Mycobacterium bovis
EGVVVHNC

(19) United States
(12) Patent Application Publication
(10) Pub. No.: US 2012/0183988 A1
(43) Pub. Date: Jul. 19, 2012

(54) INTEIN-BASED FLUORESCENT BIO-CIRCUIT FOR VITAMIN D DETECTION

(76) Inventors: G. Tayhas R. Palmore, Providence, RI (US); Vince Siu, Providence, RI (US)

(21) Appl. No.: 13/387,207

(22) PCT Filed: Jul. 30, 2010
(86) PCT No.: PCT/US10/43841

§ 371 Date: Mar. 19, 2012

Related U.S. Application Data
(60) Provisional application No. 61/229,967, filed on Jul. 30, 2009.

(51) Int. Cl. G01N 21/64; C07K 14/35

(52) U.S. Cl. 435/29; 530/350

(57) ABSTRACT

This invention is directed to an intein-based vitamin D fluorescent biosensor that accurately and rapidly measures 1α,25-hydroxyvitamin D3. Measurement at picomolar quantities is noted. Particular reference is made to the use of Escherichia coli (E. coli) as a sensor organism, although yeast and mammalian host organisms are also contemplated.

### PRIORITY CLAIM

This application claims benefit of U.S. Provisional Patent Application No. 61/229,967 filed on Jul. 30, 2009, which is incorporated by reference herein in its entirety.

### FIELD OF THE INVENTION

This invention is directed to an intein-based vitamin D fluorescent biosensor that accurately and rapidly measures 1α,25-hydroxyvitamin D₃. Measurement at picomolar quantities is noted. Particular reference is made to the use of Escherichia coli (E. coli) as a sensor organism, although yeast and mammalian host organisms are also contemplated.

### BACKGROUND

Vitamin D₃, cholecalciferol, is an essential pro-hormone that is obtained through the diet or produced photochemically in the skin. Vitamin D₃ is hydroxylated in the liver to become 25-hydroxyvitamin D₃ (25(OH)D₃), which is further hydroxylated in the kidney by a membrane-bound enzyme cytochrome P450 27B1 (CYP27B1) to become 1α,25-dihydroxyvitamin D₃ (1α,25(OH)₂D₃). This active vitamin D metabolite is reported to be involved in maintaining calcium and phosphate levels in the blood and plays a critical role in immune regulation¹. Typical vitamin D assays measure 25(OH)D₃, due to its prolonged serum half-life (ca. 3 weeks)², however, the measurement of 1α,25(OH)2 D₃ is important for patients with chronic kidney failure or detection of CYP27B1 enzymatic dysfunction. Clinically, low vitamin D levels are associated with a growing number of diseases including secondary hyperparathyroidism³, congestive heart failure⁴ and prostate, breast and colon cancer⁵. Moreover, it has been reported that severe vitamin D deficiency causes rickets in children and osteomalacia in adults⁵.

Current methods for 1α,25(OH)₂D₃ detection include in vitro competitive protein binding assays (CPBA) and in vivo transcription based assays. CPBA is based on the principles of competitive binding, where new ligands are introduced to displace a labeled ligand from a receptor's ligand binding domain (LBD). A highly purified receptor is required, and earlier versions of this displacement assay required the use of radiolabeled isotopes⁶. The in vivo transcription based assay is a frequently used technique that uses cultured mammalian cells transfected with a receptor construct and a reporter gene. Typically, a nuclear receptor's LBD is fused to a Ga14 DNA-binding domain (DBD) to create chimeric receptor. The concentration of 1α,25(OH)₂D₃ is determined by measuring the activity of the reporter gene formed upon ligand binding to the receptor. Current drawbacks to this method include long incubation time (6 to 7 days) and inconsistent results depending on cell line and assay conditions used⁷. High performance liquid chromatography (HPLC) is the current gold standard for measuring vitamin D concentrations. Although accurate, performing an HPLC analysis is labor intensive, requiring complex sample preparation and large sample volumes⁸.

### SUMMARY OF THE INVENTION

This invention comprises an intein-based fluorescent biosensor that measures receptors from the group consisting of nuclear hormone receptors, orphan receptors and non-hormone receptor. In a particular embodiment, this biosensor is an intein-based vitamin D fluorescent biosensor.

The instant invention offers a fluorescent whole cell vitamin D sensing assay. The instant invention includes an intein-based vitamin D fluorescent biosensor that measures 1α,25-hydroxyvitamin D₃. In one embodiment the further comprising sensororganisms selected from the group comprising Escherichia coli (E. coli), yeast, and mammalian host organisms.

### BRIEF DESCRIPTION OF THE DRAWINGS

FIG. 1 presents sequence information on an intein.

FIG. 2 is a diagrammatic representation of what is believed to be the intein shape change that triggers the splicing event.

FIG. 3 is a schematic of an intein-based vitamin D fluorescent biosensor that measures 1α,25-hydroxyvitamin D₃.

FIG. 4 is a schematic of two constructs embodying the technology. Examples of genetic constructs for vitamin D detection: (I) genetic sequences for hVDR and pEGFP are removed from generic amplification plasmids and subsequently ligated to pET26b expression plasmid to yield hVDR-pEGFP-pET26b construct; (II) separation of pEGFP-intein from ER-LBD so that hVDR-LBD can be inserted into pEGFP-intein sequence to yield hVDR-LBD-pEGFP-intein biocircuit.

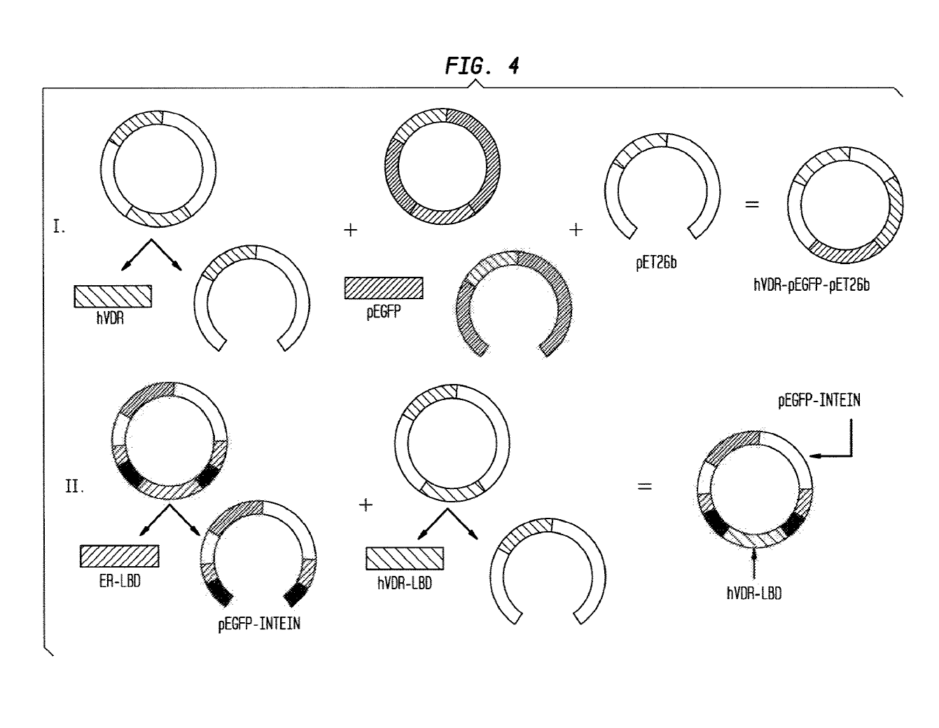

### DETAILED DESCRIPTION OF THE INVENTION

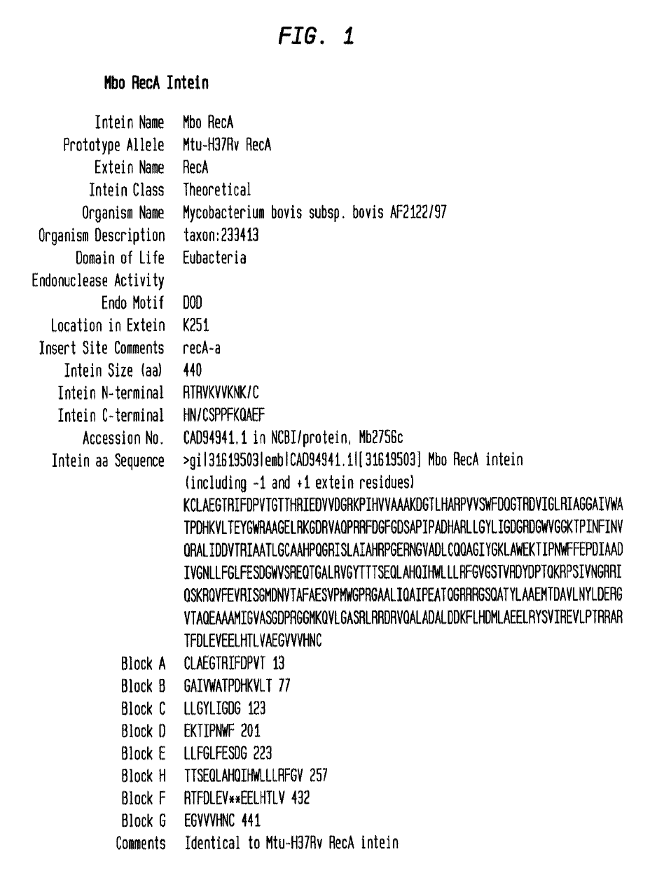

A biosensor is a measurement device that is composed of a biological sensing component that recognizes a chemical or physical change. A biosensor is typically coupled to a transducing element that produces a proportional and measurable signal in response to this change. The instant disclosure presents a synthetic genetic circuit that variously: (1) measures 1α,25(OH)₂D₃ levels in patients' serum rapidly and efficiently; (2) determines the binding affinities of synthetic vitamin D drug analogs to the vitamin D receptor; and (3) classifies the agonistic and antagonistic effects of synthetic analogs on 1α,25(OH)₂D₃-dependent genes. In one embodiment, this device comprises a vitamin D receptor (VDR) inserted within a Mycobacterium tuberculosis RecA intein that is capable of splicing a functional reporter gene such as the green fluroescent protein (GFP) upon binding of the ligand to the receptor. Reference is made to the RecA intein sequence information of FIG. 1.

The amount of functional GFP expressed is quantified spectrophotometrically and correlated to the amount of 1α,25(OH)₂D₃ binding to the VDR in a dose-dependent manner. This mechanism is conveniently expressed in an E. coli host. Those skilled in the art will understand that this mechanism is also suitable for expression into yeast and mammalian host systems.

Additionally, due to the modularity of the genetic circuit, intein-based biosensors can be modified to target other nuclear hormone receptors (NHRs), orphan receptors (ORs) and non-hormone receptors. Current reports disclose 48 human nuclear receptors encompassing 28 ligand binding sites (assuming that the isoforms of specific receptors will bind to the same natural ligand)⁹. Without being bound by any particular theory it is believed that from this set of 28, eight are classical hormone NHRs and 20 are ORs, most of which are waiting to be deorphanized. The benefit of using an intein-based bio-circuit as a deorphanization strategy is that the system is highly sensitive to minimal binding activity and has the potential to identify orphan receptor ligands that elucidate novel metabolic pathways and potential drug targets. In the interest of clarity it will be understood that am orphan receptor is an apparent receptor that has a similar structure to other identified receptors but whose endogenous ligand has not yet been identified.

System Design

1. Overview

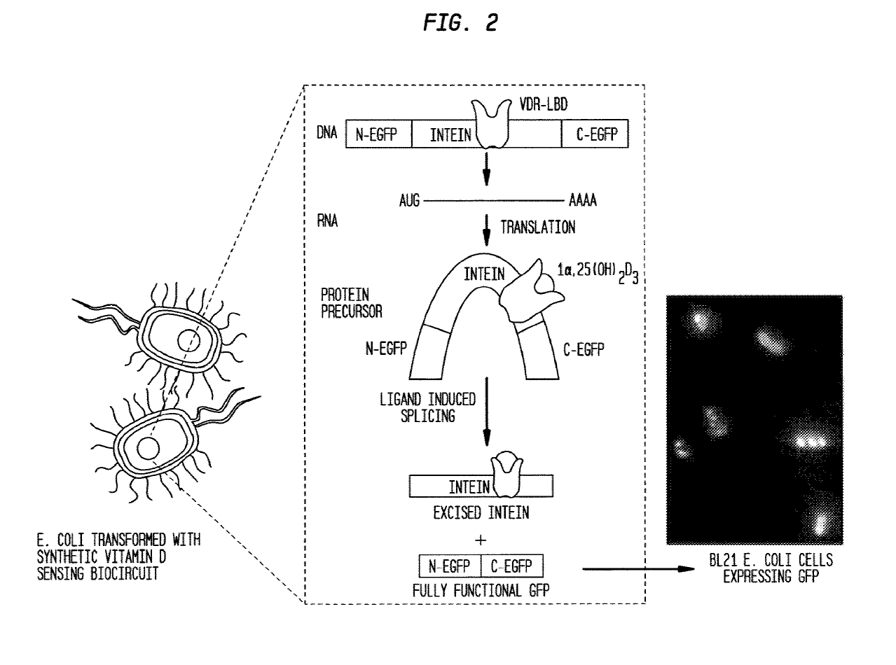

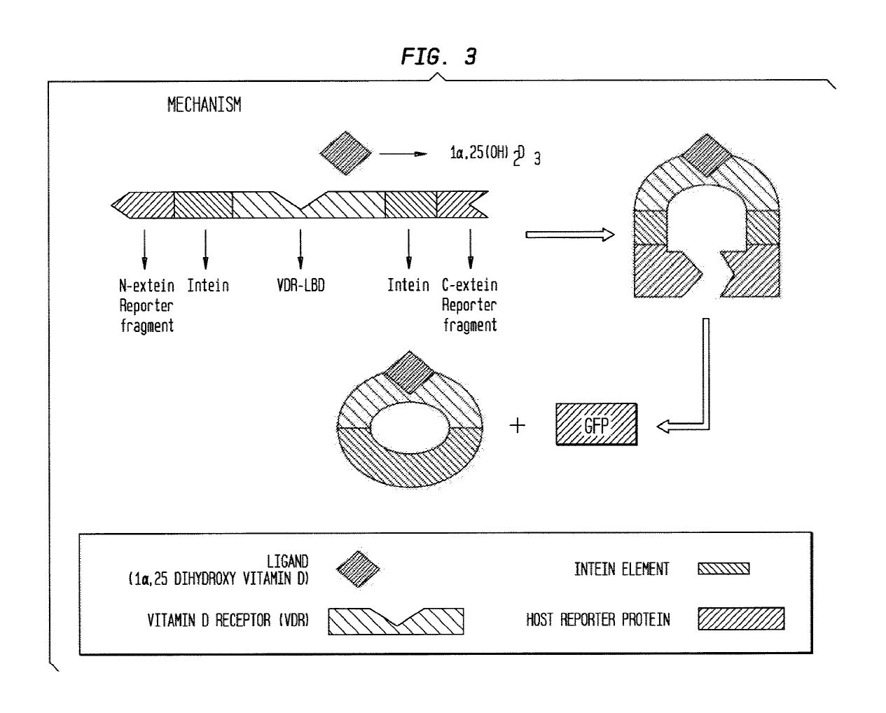

In one embodiment, an E. coli such as BL21 strain is used as a host for the vitamin D biosensor. A chimeric fusion is assembled by replacing the dispensable homing endonuclease domain of the M. tuberculosis RecA intein with the LBD of the VDR. To generate reporter activity, the intein(N)-VDR-intein(C) is inserted into Aequorea victoria enhanced green fluorescent protein (EGFP) at amino acid residues 107 and 108 as depicted in FIG. 2. Without being bound by any particular theory it is believed that when the VDR-LBD binds 1α,25(OH)₂D₃ or other small molecules, a conformation change is induced in the receptor. This change causes a shift in the two-intein fragments, facilitating the start of reconstituting the GFP extein fragments. Through a self-cleaving mechanism, the RecA intein is capable of producing a functional GFP that is completely excised from the rest of the circuit. Thus, a dose-dependent calibration will be generated by varying different concentrations of 1α,25(OH)₂D₃. FIG. 3 offers graphic representations of the methodology.

FIG. 2 is a diagrammatic representation of what is believed to be the intein shape change that triggers the splicing event. Reference is further made to Ozawa et al., “Protein Splicing-Based Reconstitution of Split Green Fluorescent Protein for Monitoring Protein-Protein Interactions in Bacteria: Improved Sensitivity and Reduced Screening Time,” Anal. Chem. 73, 5866-5874 (2001), the teachings of which are incorporated herein by reference.

2. Choosing an Appropriate Host

The BL21(DE3) strain of Escherichia coli is a suitable host for this vitamin D biosensor. A noted quality of this strain is that it contains a stable lambda lysogen which carries the T7 gene. The T7 gene encodes for the T7 RNA polymerase under control of the lac operator/promoter. The T7 RNA polymerase E. coli is a well-characterized model organism for use in biotechnological applications. Other strains are useful.

3. Choosing the Expression Vectors

In particular embodiments, all subcloning plasmid manipulations are performed using E. coli DH5α strain, while the final constructed bio-circuit is expressed in BL21(DE3) cells using a pET plasmid. Commercially available pET¹⁰ plasmids are under the control of a strong bacteriophage T7 promoter, which gives high copy and controllable level of expression of target genes in cells the bear the T7 RNA polymerase gene. A high copy number for expression in biocircuit is useful to reduce the output response time.

4. Nuclear Receptor Structure

Nuclear receptors including ORs often share a common modular structure comprised of multiple domains with specific functional features. DNA-binding domains (DBD) typically harbor two C4-zinc finger motifs that facilitate binding to specific hormone response elements (HREs) located upstream of the receptor's targeted genes¹¹. Generally in HREs, two hexanucleotide half-sites are arranged either as direct or inverted repeats separated by a short variable sequence (0-6 nucleotides) that is specific to receptor dimerization. NHRs can homo-dimerize with an identical partner or hetero-dimerize with RXRs on the HREs¹². Besides ligand recognition, the C-terminal LBD contains the hydrophobic ligand binding pocket (LBP) and the ligand-dependent transactivation function (AF-2). The N-terminal AF-1 (activating function-1) domain is capable of initiating ligand-independent functions. Specifically, the LBD of the VDR is located at amino acid residues 108-427¹³.

5. Intein Structure and Protein Splicing Mechanism

Inteins (internal protein), are internal polypetide sequences that have the ability to post-translationally cleave peptide bonds, to ligate protein fragments and to generate carboxy-terminal alpha-thioesters¹⁴. Note is made of about 454 intein sequences listed in InBase, the intein database (http://www.neb.com/neb/inteins.html) as of Apr. 15, 2009, the teachings of which are incorporated by reference herein in their entirety. Inteins in the protein precursor have conserved motifs consisting of two intein fragments sandwiching a disposable homing endonuclease domain. Studies report that the intein and endonuclease domains have distinctly separate functions. A mini-RecA intein (211 aa) with its natural endonuclease domain excised has been reported fabricated. It has further been reported to possess the same level of splicing activity as wild-type RecA intein¹⁵.

Protein splicing is an intramolecular process that requires no exogenous cofactors or sources of metabolic energy. Information necessary for protein splicing resides in the intein polypeptide plus the first C-extein amino acid residue. Protein splicing involves four coupled nucleophilic displacements. The first is a nucleophilic attack by the activated side chain of the first intein residue (Ser, Thr, or Cys) on the preceding carbonyl group, resulting in an acyl shift of the N-extein to the side chain of the first intein residue. Second is a transesterification reaction in which the hydroxyl or thiol group of the first C-extein residue (Ser, Thr or Cys) attacks the (thio)ester linkage, forming a branched intermediate with two N termini. Third is cleavage of the amide linkage at the intein C terminus by Asn or Gln cyclization to release the intein. Fourth is spontaneous rearrangement of the (thio)ester linkage between ligated exteins to form a native peptide bond¹⁶.

6. System Calibration and Characterization

Upon successful protein splicing induced by incubation of 1α,25(OH)2D₃ with the cell, the next step in an initial protocol is to calibrate the overall system. Varying concentrations of 1α,25(OH)2D₃ are incubated with the engineered cells for a fixed time and the fluorescent intensity is measured spectrophotometrically at wavelengths of 488 nm and 509 nm. An analogous process is used for other nuclear hormone receptors (NHRs), orphan receptors (ORs) and non-hormone receptors

7. Discussion

The use of a whole cell engineered E. coli for measuring vitamin D serum levels is a much more rapid, highly specific and sensitive assay compared to current detection methods. This whole-cell approach eliminates the need for laborious and expensive enzyme extraction or antibody development. Using a synthetic biology approach, an intein-based biosensor measures either the concentration of 1α,25(OH)2D₃ or the binding properties of various vitamin D analogs to the VDR. This system offers miniaturization and portability of the sensor. In one embodiment, health providers incubate a patient's serum sample with a packet of vitamin D sensing E. coli inside a multi well (e.g. 96) plate and obtain a read-out. This approach allows for rapid, accurate and low cost measurement that reduces the cost and time associated with diagnosing vitamin D deficiency.

8. References
1. Norman, A. W. 1998. Sunlight, season, skin pigmentation, vitamin D, and 25-hydrovitamin D: integral components of the vitamin D endocrine system. Am. J. Clin. Nutr. 67: 1108-1110.
2. Haddad, J. G., Stamp, T. C. B. 1974. Circulating 25-hydroxyvitamin D in man. Am. J. Med. 57: 57-62.
3. McKenna, M. J., Freaney R. 1998. Secondary hyperparathyroidism in the elderly: means to defining hypovitaminosis D. Osteoporos. Int. 8:S3-6.
4. Zitterman, A., Schulze-Schleithoff, S., Tenderich, G., Berthold, H. K., Korfer, R., Stehle, P. 2003. Low vitamin D status: a contributing factor in the pathogenesis of congestive heart failure. J. Am. Coll. Cardiol. 43: 105-112.
5. Bouillon, R. Okamura, W. H., Norman A. W. 1995. Structure-Function Relationships in the Vitamin D Endocrine System. Endocrine Reviews. 16(2): 200-257.
6. Jones, S. A., J. J. Parks, et al. (2003). “Cell-free ligand binding assays for nuclear receptors.” Nuclear Receptors 364: 53-71.
7. Zacharewski, T. (1997). “In vitro bioassays for assessing estrogenic substances.” Environmental Science & Technology 31(3): 613-623.
8. Zerwekh, J. E. 2004. The measurement of vitamin D: analytical aspects. Ann. Clin. Biochem. 41: 272-281.
9. Willson, T. M. and J. T. Moore (2002). “Genomics versus orphan nuclear receptors—A half-time report.” Molecular Endocrinology 16(6): 1135-1144.
10.Causey, J. 1998. pET: Expression Vectors System. <http://www.bio.davidson.edu/Courses/Molbio/MolStudents/spring2003/Causey/pET.html>. Accessed 2008 December 11.
11. Olefsky, J. M. (2001). “Nuclear receptor minireview series.” Journal of Biological Chemistry 276(40): 36863-36864.
12. Mangelsdorf, D. J. and R. M. Evans (1995). “The Rxr Heterodimers and Orphan Receptors.” Cell 83(6): 841-850.
13. Feldman, D., Glorieux, F H, Pike, J W. Vitamin D. 1997. Academic Press. USA.
14. Shingledecker, K., S. Q. Jiang, et al. (1998). “Molecular dissection of the Mycobacterium tuberculosis RecA intein: design of a minimal intein and of a trans-splicing system involving two intein fragments.” Gene 207(2): 187-195.
15. Noren, C. J., J. M. Wang, et al. (2000). “Dissecting the chemistry of protein splicing and its applications.” Angewandte Chemie-International Edition 39(3): 450-466.

The teachings of all references cited herein are incorporated by reference in their entirety.

1. An intein-based fluorescent biosensor that measures receptors from the group consisting of nuclear hormone receptors, orphan receptors and non-hormone receptors

2. The biosensor of claim 1 wherein said sensor is an intein-based vitamin D fluorescent biosensor

3. An intein-based vitamin D fluorescent biosensor that measures 1α,25-hydroxyvitamin D₃.

4. The biosensor of claim 3 further comprising sensororganisms selected from the group comprising Escherichia coli (E. coli), yeast, and mammalian host organisms.
